(19) United States
(12) Patent Application Publication
(10) Pub. No.: US 2011/0245707 A1
(43) Pub. Date: Oct. 6, 2011

(54) PORTABLE STROKE MONITORING APPARATUS

(76) Inventors: James Sherman Castle, Highland Park, IL (US); Jonathan Axelrod, New York, NY (US)

(21) Appl. No.: 13/074,061

(22) Filed: Mar. 29, 2011

Related U.S. Application Data
(60) Provisional application No. 61/320,024, filed on Apr. 1, 2010.

(51) Int. Cl. A61B 5/0476

(52) U.S. Cl. 600/544

(57) ABSTRACT

A method for monitoring a patient to detect the onset of a stroke, the method executed at least in part by a control logic processor, obtains, from one or more electrodes on the patient's scalp, at least a first brain wave signal pattern and a second brain wave signal pattern from the patient. The system compares at least the first brain wave signal pattern to the second brain wave signal pattern and reports a stroke, storing, in an electronic memory, a record indicating the time of the stroke according to the comparison.

### CROSS REFERENCE TO RELATED APPLICATIONS

Priority is claimed from Provisional U.S. Patent Application Ser. No. 61/320,024, entitled “A system and apparatus designed to monitor a patient for the purpose of detecting the presence of an acute stroke and alerting appropriate personnel of a stroke occurrence” by James S. Castle et al., filed on Apr. 1, 2010, the disclosure of which is incorporated by reference in this application.

### FIELD OF THE INVENTION

This invention generally relates to medical monitoring and detection devices and more particularly relates to apparatus and methods for monitoring a patient to detect symptoms of ischemic stroke activity.

### BACKGROUND OF THE INVENTION

Strokes are the third-leading cause of the death and the leading cause of disability in the United States, with an estimated 700,000 strokes occurring each year in the U.S. Globally, this number is many times larger. While some of these strokes occur with little or no apparent warning, many are experienced by patients known to be at higher risk of a stroke. If these at-risk patients could be effectively monitored so that the onset of a stroke were rapidly identified, there would be an opportunity to prevent or reduce the severity of a significant number of strokes each year.

Strokes can be categorized by type, based on their location and nature. Some, such as intracranial hemorrhages, are unpredictable; others, such as small deep strokes, are frequently too minor to notice. However, a significant portion of strokes are large, surface-based, and ischemic, meaning they are caused by a blockage in a large artery. Such a potentially debilitating stroke has the potential to be identified at the time of occurrence or shortly thereafter. This potential for rapid detection is medically critical since the time elapsed from stroke onset until medical treatment is the most significant variable factor affecting successful treatment of an acute stroke. In short, medical treatment administered quickly is critical for either preventing or significantly ameliorating the medical impact of a stroke on a patient.

There are currently a variety of known conditions which place a patient at higher risk of experiencing such an ischemic stroke. One such condition which often harbingers a future stroke is a Transient Ischemic Attack (TIA). A TIA is defined as a transient blockage of an artery supplying blood to an area of the brain or the eye that causes symptoms lasting less than twenty-four hours. The American Stroke Association (ASA) estimates that 200,000-500,000 cases of TIA occur each year in the United States. The ASA also estimates that anywhere from 10-15% of TIA patients will have a stroke within three months of their initial TIA, and that half of these strokes will occur within the first 48 hours after a TIA. Such patients are therefore known to be at-risk and to benefit from close observation for the potential onset of a stroke. As a result, even though a TIA itself generally causes no lasting damage, it offers an opportunity to predict and potentially prevent future, severe strokes.

Several other conditions are also known to place a patient at higher risk for a stroke. These conditions include, but are not limited to: pre-operative patients who must refrain from taking their blood thinners because of an upcoming surgery; patients undergoing cardiac catheterization who have a propensity for clot formation or cholesterol embolization; and patients who have recently undergone surgery or other intervention on an artery supplying blood to the brain. Similar to patients who have experienced a TIA, these other patients are candidates for close observation to enable the early identification of a stroke's onset.

Under the current standard of care, after the identification of a condition that places a patient at-risk for a stroke, there remains little that can be done to automate the monitoring of the patient. To clarify, monitoring a patient for a stroke herein refers to documented observation of a patient's neurological status. At the present time, monitoring is limited to human observation of an at-risk patient that can be validated through neurological exams.

The current visual or observation-based monitoring procedures are used in large part because there is no device, automated monitor, or automated monitoring system to our knowledge in current use to detect or to provide early warning for a stroke. Moreover, strokes as events are not observable through the various automated monitoring devices conventionally deployed in hospitals, such as cardiac or respiratory monitors.

Current best-practices for monitoring a stroke mainly consist of manually checking a patient's alertness and neurological function at periodic intervals. The monitoring process requires that a person make a visual check of a patient's neurological signs (such as new onset weakness, numbness, difficulty with speech, or changes in vision) that indicate the likely onset of a stroke. The person performing the observation then records a rough estimate as to the exact time the symptoms could most likely have started and contacts physicians for immediate treatment. There are no clear guidelines as to how long or how frequently a TIA patient or other at-risk patient must remain in the hospital, nor how frequently neurological observations should continue. Given the expense of a hospital stay, most patients cannot be kept in the hospital for the sole purpose of monitoring for a stroke.

Once a patient is placed under stroke observation, the frequency and duration with which these neurological checks are performed can vary greatly. In a hospital, visual monitoring for stroke detection generally occurs every two to four hours and is performed by a nurse or physician. The precise protocol for observation varies by local hospital policy and can be affected by other factors including the work-load of the covering nurse, whether or not the patient is sleeping and wishes not to be disturbed, and various other conditions generally not related to the medical necessity of the situation. Even facilities with the best nursing coverage suffer from inconsistent monitoring because of the practical realities of dealing with multiple patients, some of whom may require urgent care or treatment.

Home observation is generally less reliable in accurately determining the presence and timing of neurologic changes and less consistent because the care-givers are often not medically trained. Night-time is of particular concern, since both the patient and care-giver are likely to be sleeping and therefore unaware of the onset of a stroke.

Further complicating the accurate identification of the onset of a stroke is the impact of the stroke on the average patient. The vast majority of ischemic strokes are painless and therefore there is no way to accurately identify and record the time of the onset of a stroke based on the patient's own reporting. In addition, because of the mental and physical disabilities that often come with a stroke, many patients will not be able to alert anyone to their new symptoms, or may not even be aware that they are having a stroke.

Timely recognition that a stroke has occurred is a critical factor in determining appropriate stroke treatment; however, this determination is often extremely difficult to make. Unless the patient is coherent enough to be able to describe exactly when their stroke started, or unless a care-taker or nurse happens to be watching them at the moment their stroke started (both of which are uncommon), the current standard of care for determining when a stroke began is to use the “time last seen normal,” meaning the time that the patient was last witnessed to appear neurologically normal. This time estimate of onset can often be hours before the event occurred, making some patients ineligible for treatments that might be available if accurate onset could be determined. For example, strokes that occur during sleep, often called “wake-up strokes”, frequently provide no interventional treatment options for doctors since the “time last seen normal” can often be as much as eight hours or longer before the patient wakes up with a perceptible neurological deficit.

The current practice, visual monitoring patients at regular intervals, almost always results in a time lag between the onset of a stroke and the communication to medical personnel that the stroke has occurred, even in situations where the neurological monitoring is accurate and timely. In other cases where the monitoring is less reliable, the time delay can be much worse. This time lag results in delay in administering medical treatment, and the medical damage caused by a stroke is often exacerbated because of this delay. A well known expression among stroke physicians is that “Time is Brain”: the sooner one acts to treat a stroke, the more likely an improved outcome for the patient. The time window for acute stroke treatment is short and has a steep slope of decreasing effectiveness with time. Timeliness is important both because of the damage caused by the blockage as well as the time-critical nature of current interventional options. The lack of automated monitoring has therefore created an acute need for a solution to this problem.

The set of requirements surrounding the administration of Tissue Plasminogen Activator (TPA) is one major reason timely identification of stroke onset is so critical. Intravenously administered TPA is currently the only Food and Drug Administration (FDA) approved medicine for acute stroke treatment. Its introduction has been a major advancement in successfully treating strokes. For example, in one study based on a meta-analysis of TPA trials in the March 2004 edition of The Lancet, patients receiving TPA within 90 minutes of their stroke had an odds ratio of 2.80 for favorable outcome at 3 months when compared to patients receiving placebo.

However, TPA is currently only approved for use within three hours after the onset of a stroke. This is particularly problematic since the average patient under stroke observation is only checked every several hours, meaning there is a strong likelihood that even a patient under a specified stroke watch will not have a stroke identified in time for TPA to be administered, despite observation protocol having been followed perfectly. Further, based on an average eight-hour sleep cycle, one-third of all strokes occur during sleep and are particularly unlikely to be detected.

The reason TPA must be withheld if the time of onset is not clearly known relates to the risks in giving a powerful thrombolytic or “clot-buster” to a patient with a stroke. The longer the time from stroke onset, the higher the risk. Even administration of TPA within 3 hours of stroke onset carries a 3% fatal hemorrhage rate based on the NINDS trial by which TPA gained FDA approval. In addition, TPA becomes less effective at reversing the effects of a stroke as time passes from the stroke onset.

Because of the difficulty in identifying the time of stroke onset, TPA is administered to only a small portion of the potential candidate patients. The February 2006 issue of the Journal of Neurology estimated that only 2% of stroke patients receive TPA. That same article noted that in 35% of all stroke patients TPA was withheld because the treating clinician had no clear indication of the time that the stroke began. Further, the journal Stroke in its March 2009 edition estimated that 25% of all stroke patients wake up with their stroke, and are therefore ineligible for TPA. Using 700,000 cases per year in the U.S. as an estimate for the total number of strokes, these studies imply that somewhere between 175,000 and 245,000 patients are not receiving TPA annually in the U.S. because the time of onset of their strokes was not known. Thus, TPA is currently under-utilized because of the limitations of current stroke monitoring practices; its wider use would result in a dramatic improvement for the expected outcome for many stroke patients.

There are other treatment options for strokes, including several FDA approved medical devices, but use of the devices is also highly dependent on knowing the exact time that a stroke started. These devices include the Merci Retriever device (Concentric Medical, Mountain View, Calif.—FDA approved device 2004) and the Penumbra System (Penumbra, Inc., Alameda, Calif.—FDA approved device 2008). These devices are an option for use within 8 hours of stroke onset.

The current best-practice for monitoring patients at risk of a stroke creates substantial delay in identifying and treating a new stroke. This delay often leads to the complete withholding of critical medical treatment and almost always results in the killing of additional brain cells. There is currently no device, automated monitor, or automated monitoring system for detecting an acute stroke. As a result, there is a need for an electronic or automated monitoring system or device that could be used to monitor a patient identified as a high-risk candidate for a stroke.

There are automated devices for medical monitoring of patients, but for conditions often unrelated to the onset of a stroke. Such devices include cardiac monitors, which monitor a patient's heart activity, and can be either connected to a fixed hospital monitoring system or worn as a portable monitor by a patient. They also include devices which monitor a patient's respiratory activity. Such devices may detect changes in a patient's general medical condition, but lack a direct connection in identifying the onset of a stroke.

There exist certain other devices that monitor a patient's brain activity, and that, subject to analysis and interpretation by a skilled practitioner, can provide evidence that a stroke has occurred. However, these devices are not typically useful for detecting the actual onset of a stroke as it occurs. Current equipment which monitors moment-to-moment brain activity exists in the form of brain electrical monitoring equipment, otherwise known as an Electroencephalogram (EEG). An EEG machine consists of a device which records brain activity using leads which attach from the device to electrodes which are placed on a patient's scalp.

EEGs were developed primarily for seizure detection and their use has remained largely limited to this purpose, with the notable exception of their use in detection of sleep stages in sleep studies, and their design has been optimized for this purpose. Seizure monitoring with an EEG is conducted by placing electrodes non-invasively on the scalp in a distribution designed to cover significant surface areas on both sides of the brain. The EEG identifies changes in the electrical activity in the surface of the brain to determine if seizure activity is occurring and the leads are therefore spread out to cover as much of the brain surface as possible. For example, the leads are arranged to detect the spread of abnormal epileptic activity across the brain surface, not placed to monitor the vascular points that would be useful in detecting a stroke. A common configuration, for example, includes 21 leads placed on a patient's head. After the EEG recording is made it is then reviewed by a physician skilled in EEG interpretation with the goal of determining the existence of seizure activity.

The EEG lead configurations currently in use also limit the development of more portable EEG designs, since the standard lead configuration is complex, too cumbersome for easy use in a portable or wearable stroke monitoring device. Some more portable epilepsy monitors exist, but they can generally be worn only for a relatively short time due to their large number of leads and cumbersome accompanying equipment.

It is instructive to note that the EEG is not the preferred diagnostic tool for stroke detection. In conventional practice, computerized tomography (CT) imaging or magnetic resonance imaging (MRI) are the preferred tools for accurately diagnosing strokes. Due to factors of availability, timing, and scheduling, however, these tools are impractical for timely detection of stroke occurrence; by the time positive results can be obtained with this equipment, most patients are well beyond the time limits allowable for TPA treatment.

Relatively recently, there has been a limited introduction of reduced-electrode EEG devices, designed to be more portable. These have generally been designed for usage by mobile or first-responder medical personal, for example EMTs or battlefield medics, to identify seizure or significant trauma. These more portable devices are designed to be used for a short period of time as an aid for rapid diagnosis of an individual patient, not for on-going monitoring and not optimized for stroke detection. They are not designed to be worn or carried by a patient, but require that the patient be stationary and seated at, or lying near, the equipment. Thus, these devices are not designed to be used by an individual patient for an extended period of time. Further, because they are merely intended to acquire and display or store the needed signals for assessment, these systems are not designed with the processing logic needed to detect stroke onset in at-risk patients. Other portable EEG devices have been used for seizure monitoring, but these devices utilize the full electrode montage, and because no reliable seizure detection algorithm technology exists, are limited to interpretation only by the retrospective review of a trained physician.

A number of solutions have been proposed for utilizing the electroencephalogram for stroke diagnosis in patients with clear underlying neurologic disease, but not for the purpose of tracking a person to monitor for a possible future event. For example, U.S. Pat. No. 7,471,978 to John et al. uses passive and evoked potential recordings for the purpose of detecting stroke victims from those with other diseases that can cause decreased responsiveness.

Also, the use of some derivation of EEG technology for stroke diagnosis, either by passive or evoked potential recording, is described in U.S. Pat. Nos. 7,231,245 to Greenwald et al., 7,024,238 to Bergethon, 6,985,769 to Jordan, and 4,608,635 to Osterholm, as well as in US Patent Applications 2009/0112117 by Rewari and 2004/0077967 by Jordan. None of these other examples directly address the problem of monitoring a high risk patient to detect the actual onset of a stroke. Most of these other approaches seek to stimulate brain function at a specific point in time in order to determine the current status of a patient and to assess whether or not a stroke has already occurred. They are also not designed to use EEG capabilities to provide a portable monitoring device and system designed for medium or long-term use by patients to detect changes to brain waves and identify and record the onset of a stroke as it occurs. They do not evaluate patients against their own baselines, use symmetry to monitor patients, or monitor vascular points.

Other examples of medical monitoring devices address the problem of monitoring a patient's well-being in general and may provide useful information, but do not directly address the problem of stroke detection. These devices include all-inclusive body monitors that use a combination of technologies to identify any change in a patient's general condition or monitors that provide an alert if a patient has fallen or has otherwise changed physical position in some way. For example, U.S. Pat. No. 7,502,498 to Wen et al. describes an approach that may indirectly detect a stroke or other catastrophic event by monitoring for a change in the physical positioning of a patient, such as a fall, using utilities such as a 3-D camera and Global Positioning Satellite (GPS) data. It must be emphasized that such an approach does not actually attempt to identify if a stroke is in process or has occurred, but simply reports that a patient has changed physical positioning. As a result, such a device does prove particularly useful in detecting the large number of strokes that occur where the patient is already sitting down or sleeping, for example. Significantly, such devices do not utilize EEG or other technology designed to monitor the brain itself.

There are proposed monitoring solutions that attempt to identify stroke onset among other conditions, such as that described in U.S. Pat. No. 7,558,622 to Tran, which describes an all-inclusive body monitor. U.S. Pat. No. 7,558,622 describes a multi-faceted system utilizing a combination of electroencephalogram electrodes, bioimpedance electrodes, and oxygen saturation monitors placed over specific portions of the brain to help detect stroke by using evoked potentials and spontaneous power spectra recording. Such a device is not, however, particularly well-suited for short term use in a high risk patient. Rather, it is designed to be used more generally to detect a “first ever stroke” in a patient who is not at high risk, just as it seeks to find any condition that arises in a patient.

Significantly, the testing approach taught in U.S. Pat. No. 7,558,622 and more generally practiced for other types of monitoring and diagnosis typically compares an acquired EEG signal against normative or “model” data. Basically, the patient's measurements are compared against statistically developed archetypes in order to assess whether or not “normal” patterns are detected. This classical approach works well for many types of diagnostic situations, but proves to be relatively complex and is not well suited for monitoring ischemic stroke onset. In practice, it has been observed that a patient can have unique brain wave patterns that may indeed be normal for that patient, but that complicate monitoring and diagnosis when compared against idealized or statistically normalized patterns obtained from a broader patient population.

Overall, it has been found that existing and proposed solutions for patient stroke monitoring are characterized by complex equipment configurations and connection mechanisms that constrain patient movement and can interfere with normal activity and rest cycles, bulky apparatus not readily adapted to be portable, characterized by high-cost and by the requirement for constant attention from trained personnel.

There is a long-felt need for portable monitoring apparatus and methods that address the problem of ischemic stroke detection and reporting the onset of a stroke for at-risk patients.

### SUMMARY OF THE INVENTION

It is an object of the present invention to advance the art of stroke monitoring and detection for high-risk patients. The present invention addresses the shortcomings of conventional solutions described in the background section. The invention provides apparatus, system, and methods to electronically monitor a patient for a potential stroke.

The apparatus, system, and methods of the present invention detect, record, and report the onset of a stroke through the use of automated monitoring, identification, recording, and communication technologies. In operation, embodiments of the present invention detect changes in electrical impulses of the brain known to be associated with stroke onset and communicate this information to appropriate response agents.

Embodiments of the present invention provide a portable device that would provide early detection, recording, and notification of a stroke based on actual second-to-second or minute-to-minute changes in the brain's activity and offers the potential to revolutionize stroke treatment. Such early notification would allow for reliable documentation of the onset of stroke and provide an opportunity for a much larger portion of stroke patients to have their condition detected quickly enough to administer TPA or other time-sensitive medical treatments.

According to an aspect of the present invention, there is provided a method for monitoring a patient to detect the onset of a stroke, the method executed at least in part by a control logic processor and comprising:
obtaining, from one or more electrodes on the patient's scalp, at least a first brain wave signal pattern and a second brain wave signal pattern from the patient;
comparing at least the first brain wave signal pattern to the second brain wave signal pattern;
and
reporting a stroke and storing, in an electronic memory, a record indicating the time of the stroke according to the comparison.

According to another aspect of the present invention, there is provided an apparatus for detecting the onset of an ischemic stroke in a patient, comprising:
a) a brain wave signal acquisition apparatus comprising a signal acquisition circuit that is energizable to provide electrode signals acquired from each of one or more electrode pairs that are attached against the patient's scalp,
wherein each electrode pair comprises a left electrode disposed at a first position to sense a left-side signal along the left side of the scalp and a corresponding right electrode symmetrically disposed at a second position to sense a corresponding right-side signal on the right side of the scalp;
and
b) a monitoring apparatus that is in signal communication with the brain wave signal acquisition apparatus and is energizable to compare the provided left-side and right-side electrode signals from each electrode pair and to generate a warning signal according to the comparison.

These and other aspects, objects, features and advantages of the present invention will be more clearly understood and appreciated from a review of the following detailed description of the preferred embodiments and appended claims, and by reference to the accompanying drawings.

### BRIEF DESCRIPTION OF THE DRAWINGS

While the specification concludes with claims particularly pointing out and distinctly claiming the subject matter of the present invention, it is believed that the invention will be better understood from the following description when taken in conjunction with the accompanying drawings, wherein:

FIG. 1 is a block diagram of a stroke monitoring system according to an embodiment of the present invention.

FIG. 2A is a side view showing relative electrode placement on the brain.

FIG. 2B is a top view showing relative electrode placement on the brain.

FIG. 2C is a side view showing relative electrode placement on the brain in an alternate embodiment of the present invention.

FIG. 2D is a top view showing relative electrode placement on the brain in the embodiment shown in FIG. 2C.

FIG. 3 is a graph that shows example signals from electrode pairs and shows how a stroke can be detected.

FIG. 4A is a side view of a headpiece worn for stroke monitoring according to one embodiment.

FIG. 4B is a side view of a headpiece worn for stroke monitoring according to an alternate embodiment with a monitoring control unit built into the headpiece.

FIG. 5 shows front and side views of a monitoring controller unit according to an embodiment of the present invention.

FIG. 6 is a schematic diagram that shows components of a monitoring controller unit according to one embodiment.

FIG. 7 is a logic flow diagram that shows control processing for stroke detection.

FIG. 8 is a schematic diagram of a wireless stroke monitoring system according to an embodiment of the present invention.

### DETAILED DESCRIPTION OF THE INVENTION

The present description is directed in particular to elements forming part of, or cooperating more directly with, apparatus in accordance with the invention. It is to be understood that elements not specifically shown or described may take various forms well known to those skilled in the art.

In the context of the present disclosure, the use of terms such as “first”, “second”, “third”, etc., does not by itself connote any priority, precedence, or order of a component or claim element over another or the temporal order in which acts of a method are performed. These terms may be used more generally as labels to distinguish one element having a certain name from another element having the same name (but for use of the ordinal term) or to distinguish the claim elements.

In the context of the present disclosure, the term “energizable” has its standard meaning and relates to a component, device, or system that can be enabled and made operational when it is suitably connected to an appropriate power source, optionally receives an enabling signal or instruction, and is in communication with any other devices necessary for its function, such as connected to network cabling, for example. Signal communication, as the phrase is used herein, can be over wire or wireless.

Embodiments of the present invention address the long-recognized need for a monitoring device that is particularly suitable for patients who are at near-term risk of ischemic stroke and that provides:
(i) improved portability, enabling the patient to be mobile and allowing the patient to follow normal patterns of activity and rest while being monitored;
(ii) reduced size and complexity over existing instrument solutions, providing a set of measurements that are likely indicators of stroke onset;
(iii) reduced cost over conventional equipment that could otherwise be used for this purpose;
(iv) ability for remote use, so that the patient being monitored does not need to be confined to a hospital or other medical facility;
(v) ease of use, so that the device can be readily checked by the patient or other care-giver;
(vi) capability for analysis, recording, and providing alert information, electronically using on-board logic or using some combination of on-board and remotely accessed logic and using audible and visual cues; and
(vii) the ability to detect the actual onset of a stroke as it occurs.

In addition, it is noteworthy to observe that the apparatus and methods of the present invention can be used for monitoring patients who already have had a previous stroke and may already exhibit some irregularity in brain wave pattern as a result.

The Inventors have recognized that, in contrast to the approaches used for full-scale EEG equipment that employs a large number of electrodes placed along the scalp surface, a simpler approach can be applied for detection of ischemic stroke onset. By using a more limited set of electrodes, or even a single electrode, strategically positioned against the scalp of the patient, measuring signals in one or more vascular distributions, such as in a symmetric arrangement, and by employing an innovative approach to signal comparison and analysis that is more appropriate for stroke detection using a limited set of signals, the approach of the present invention is able to provide a monitoring device that more closely satisfies the characteristics and features noted in (i)-(vii) above.

Instead of attempting to acquire comprehensive brain wave data from numerous measurement points and to compare these acquired measurements against normative data as in earlier proposed solutions, embodiments of the present invention attempt to make fewer measurements with more strategic importance for stroke detection by exploiting two principles that have not been widely recognized:
(1) Measurement symmetry. The Inventors have observed that there is significant symmetry in normal brain wave patterns for signals detected by electrodes that are, correspondingly, symmetrically placed at suitable positions, such as in the regions of the major cerebral arterial distributions, along the scalp. Asymmetry of corresponding signals can be shown to be a suitable indicator of an ischemic stroke incident and can be readily detected in brain wave signal patterns.
(2) Characteristic patient brain wave signal patterns. The Inventors have also observed that each individual patent's EEG reading is unique, often with wide fluctuation in frequency and voltage of the brain waves throughout the day. This complicates the task of detecting stroke onset using conventional normative approaches. However, embodiments of the present invention use this observation to advantage, since significant changes from a patient's baseline brain wave patterns can also be symptomatic of stroke onset.

By making measurements at specific “eloquent” brain regions that relate to major cerebral arteries, a relatively quick and accurate assessment can be made for stroke onset. As noted in the background section, conventional approaches to stroke assessment are not directed to detecting the actual onset of a stroke, but are more effective in obtaining data after the fact. By comparison, the apparatus and method of the present invention provide detection of stroke onset, more accurately indicating that a stroke is actually in process or has just occurred.

Once detected, the apparatus of the present invention has the ability to alert medical personnel, a quick-response facility, and/or nearby persons to the fact that an acute stroke is occurring. This device would enable a high-risk patient to be monitored for an impending stroke even while alone or asleep. By comparison with conventional devices that obtain brain wave signals, the apparatus of the present invention uses only a small number of electrodes, typically disposed within a protective headband or other headpiece. The protective headpiece helps to contain and cover the electrodes to help allow improved patient comfort and mobility.

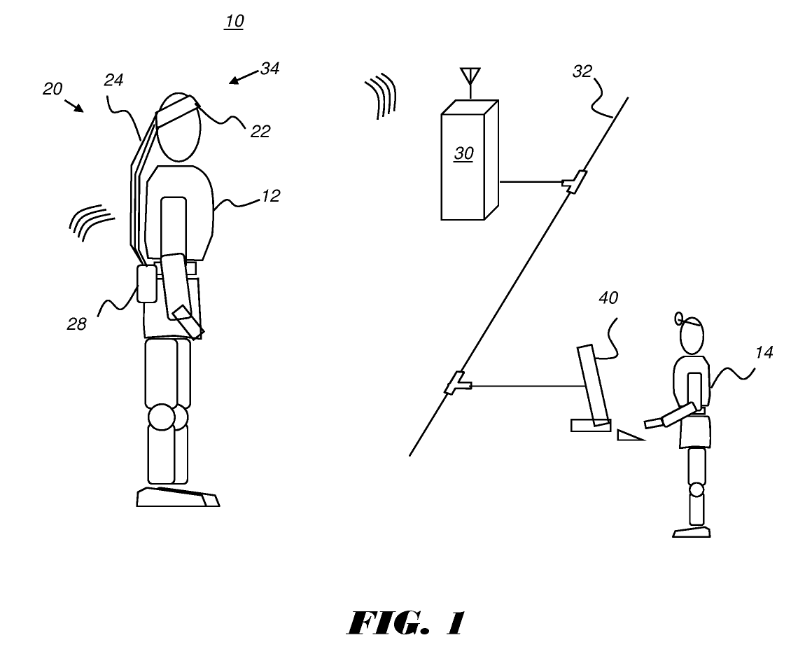

Referring to FIG. 1, there is shown a portable stroke monitoring and detection system 10 consistent with an embodiment of the present invention. A patient 12 is provided a brain wave signal acquisition apparatus 34 that includes an optional headpiece 22 that helps to maintain one or more sets of electrodes in place against the patient's scalp. A lead 24 connects each electrode to a monitoring control unit 28 that includes signal acquisition and logic components for at least a portion of the monitoring function. As part of a monitoring apparatus 20, control unit 28 is in communication with a host processor 30, such as using a wireless network connection, for example. Host processor 30, typically a networked computer or workstation, is connected to a network 32 that provides monitoring information on a display 40 so that it can be viewed by a practitioner 14. Host processor 30 also includes other functions, such as storing the received signal data from patient 12. In addition, host processor 30 may provide some portion of the automated logic for stroke onset identification.

It can be readily appreciated that the model system shown in FIG. 1 can be embodied in a number of ways without departing from the present invention, as described in more detail subsequently. For example, the optional headpiece 22 can be arranged in a number of ways. In addition, some portion or all of the control logic and reporting functions of monitoring apparatus 20 can also be executed within monitoring control unit 28 or remotely.

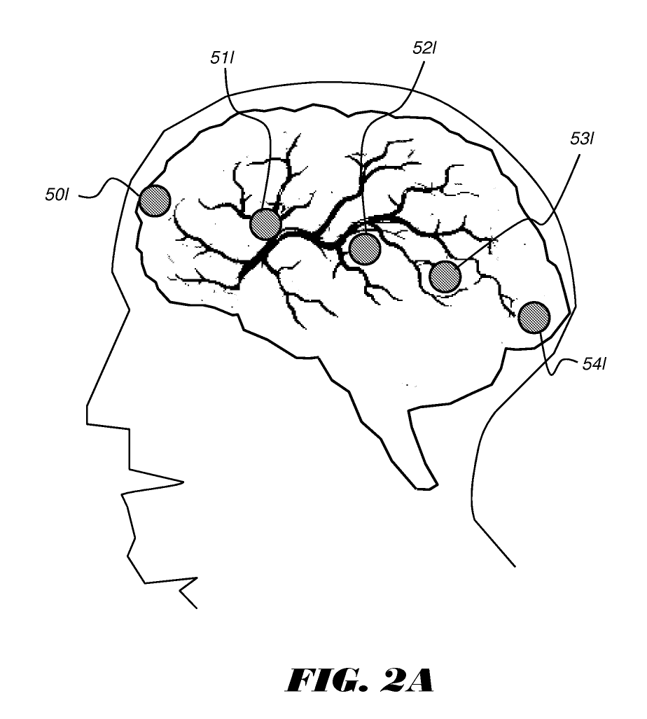

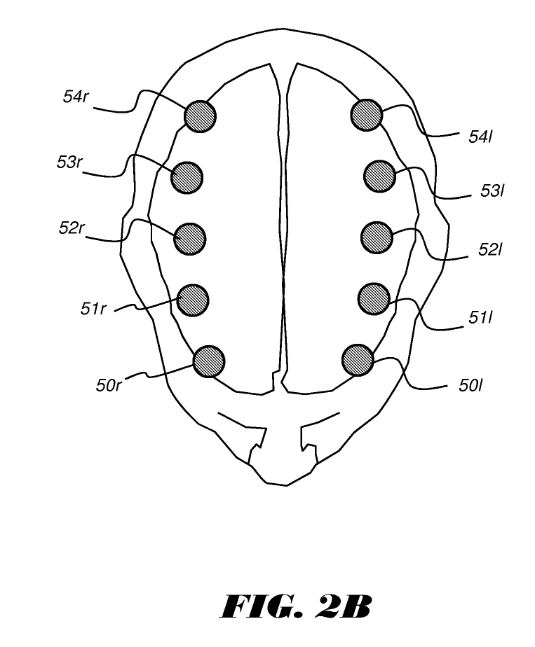

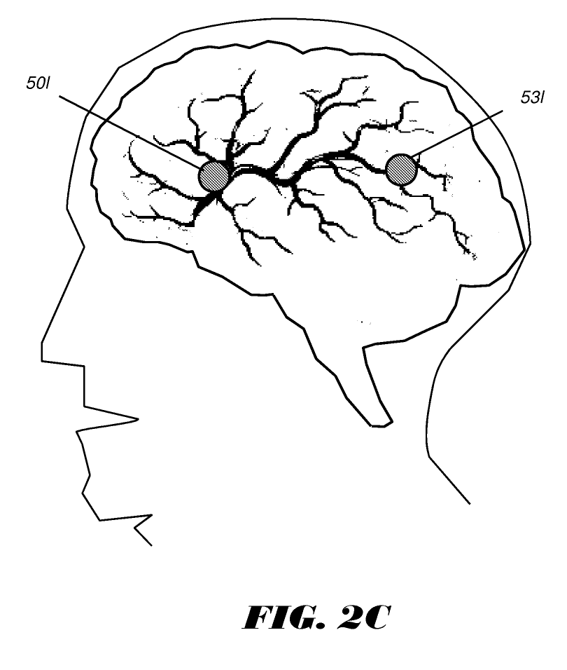

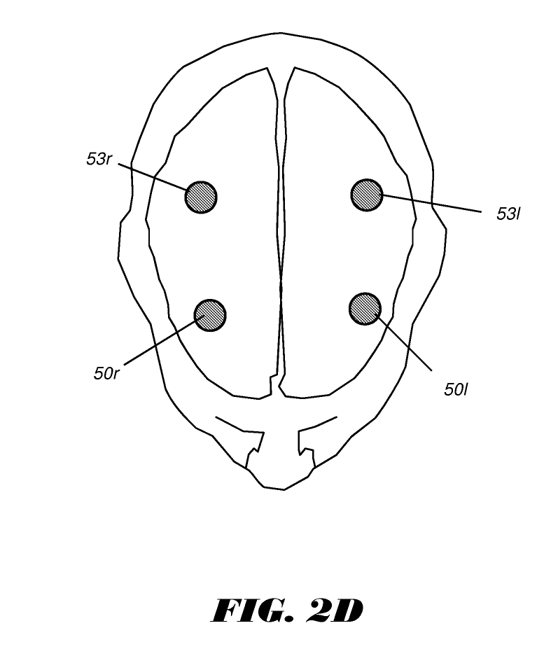

As noted earlier, embodiments of the present invention employ a limited number of paired electrodes for ischemic stroke detection as part of the stroke monitoring and detection system. The side view of FIG. 2A and top view of FIG. 2B and side and top views of FIGS. 2C and 2D show arrangements of paired electrodes 50l, 50r, 51l, 51r, 52l, 52r, 53l, 53r, 54l, and 54r, wherein the appended “l” and “r” indicate right and left electrodes respectively, and their relative placement according to one embodiment, described in more detail subsequently.

### Brain Wave Signal Patterns

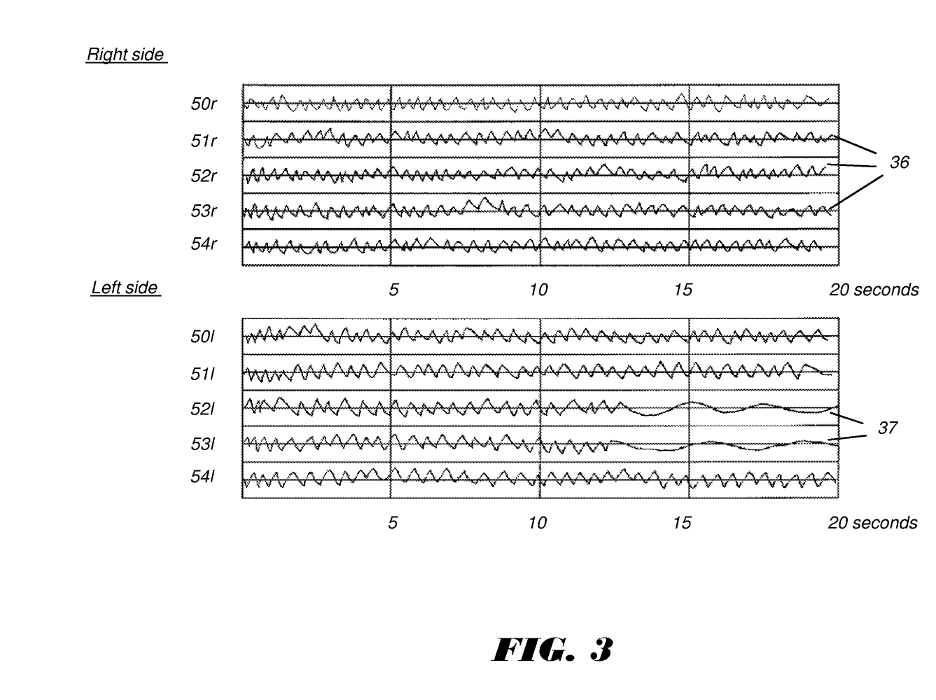

The graph of FIG. 3 shows an example recording of brain wave signal patterns 36 that track the electrical signals from the paired electrodes over time, using the electrode arrangement described with reference to FIGS. 2A and 2B. For the first 13 seconds, the graph shows generally symmetrical activity of signal patterns 36, with sudden onset of asymmetrical activity at about 13 seconds as shown at abnormal signal patterns 37. This change in activity occurs with the affected stroke region showing a sudden change in frequency and decrease in voltage amplitude on one side when compared to the normal side. Monitoring and detection system 10 of the present invention is designed to detect such a condition, so that it can be promptly reported and recorded, thus improving the chance that a patient can receive early corrective treatment for the stroke. After a positive identification of abrupt, asymmetrical brain wave signal pattern changes, such as those shown in the example of FIG. 3, the apparatus of the present invention generates a signal that communicates the change of status to the appropriate monitoring agent.

The time interval over which the brain wave signal pattern is sensed is variable and may range from a few seconds to several seconds or longer, for example, and each signal pattern may be processed in a number of ways in order better isolate the pattern from noise. Embodiments of the present invention detect stroke onset by comparing at least first and second brain wave patterns from the same patient. The first and second brain wave patterns may be obtained during different time intervals, in which case the same electrode typically obtains both first and second patterns over the same area, or may be obtained simultaneously, in which case paired electrodes, placed symmetrically, obtain the first and second brain wave patterns from left and right sides of the scalp.

As noted previously, the Inventors have observed that the two sides of the brain generally exhibit relatively symmetric electrical activity in regard to amplitude of voltage and frequency. As a result, a degree of electrical symmetry is generally stable for each individual person and a departure from this symmetric pattern represents a change in the person's brain activity. It can be appreciated that symmetry is an approximation, since even the two sides of the head exhibit at least some asymmetry in most people.

In addition to left-right symmetry, each individual can have unique characteristic baseline brain wave signal patterns. Embodiments of the present invention measure signal characteristics such as the amplitude and frequency of the patient's brain wave signal patterns, and, after establishing the individual patient's baseline brain wave signal patterns for some time period, are sensitized to changes to brain activity. A notable change in symmetry or a pronounced deviation from the patient's standard brain wave signal patterns can indicate focal brain dysfunction, likely indicating an acute ischemic stroke.

For the purposes of detecting a stroke, embodiments of the present invention compare the symmetry of signals for each electrode pair or, alternately, detect a pronounced deviation from the patient's baseline brain wave signal patterns, or may detect both asymmetry and deviation from baseline patterns. For symmetry monitoring, each electrode's signal is compared against the signal from the corresponding electrode on the other side of the scalp (right versus left) using metrics such as summated amplitude of the recorded voltage deflection over a specified time period (amplitude asymmetry) and/or the number of deflections of the voltage recording over a specified time period (frequency asymmetry). Additional data using the recorded voltage amplitudes and time comparisons may also be used as well to test for symmetry. Given that a stroke results in a persistent and quantitative change in electrical activity, the task of using an algorithm to monitor brain wave signal patterns for such an occurrence differs substantially from implementing an algorithm for seizure detection. By comparison, attempts to utilize algorithms to detect seizures have proven difficult to perfect, given a seizure's episodic nature which can often only be identified by a qualitative reading of an EEG recording.

According to one embodiment of the present invention, an algorithm on the monitoring control unit 28 analyzes the electrical activity recorded over each electrode. Monitoring control unit 28, or other logic circuitry that is in signal communication with monitoring control unit 28, then calculates the amplitude, in volts, of maximum and minimum points of deflection over a few seconds, and compares that with the signal from the corresponding electrode on the other side of the brain.

The monitoring device would also be able to calculate the number of cycles per second of deflection. A filter could be used to minimize noise from a low amplitude signal. The monitoring control unit then compares that data with the number of cycles per second on the corresponding electrode on the other side of the brain. The monitoring device is equipped with a detection system of sufficient sensitivity and specificity as to note a sustained change in brain wave signal patterns. This may be a change in the symmetry, both in voltage of deflection and number of cycles of deflection, between two corresponding electrodes on opposite sides of the brain, such as would be expected during an ischemic stroke. Alternately, this may be a difference in brain wave signal patterns taken from the same patient at different times, where the pattern acquired earlier is considered a baseline brain wave signal pattern and is obtained and stored in memory to provide information on the normal characteristic brain wave activity of that patient.

Once the algorithm has identified a condition that is believed to correspond with the onset of a stroke, whether using an assessment of asymmetry or of divergence from the baseline pattern or both, a signal is generated so that an appropriate alerting mechanism is triggered. It can be appreciated that the detection system logic can be located on monitoring control unit 28 itself or can be located on some other logic processor that is in signal communication with monitoring control unit 28, as is described in more detail subsequently.

### Electrode Placement

Consistent with an embodiment of the present invention is a novel placement of electrodes for the purpose of optimizing detection of an ischemic stroke, allowing the recording device to be easily portable. The invention utilizes a series of one or more electrodes, similar to those used in EEG equipment, that attach to the scalp and provide the detected signal for monitoring. Signal communication from the electrode uses a wired or, alternately, a wireless connection.

The electrodes record surface-level electrical activity. The electrical activity recorded by an electrode at the scalp is a small fraction of total brain electrical activity at that site, since the skull blocks most of the signal. Each scalp electrode senses the synchronous electrical activity of the multitude of neurons lying just underneath the skull at the electrode site and records this as voltage amplitude, graphed out as a function of time, as is typically performed by the EEG or other recording machine. Thus, for example, the machine that records an electrode signal graphs a curvilinear representation of both the amplitude and frequency of regional cortical (or surface) brain activity (voltage being on the “y” coordinate, time being on the “x” coordinate, as shown in the example of FIG. 3). The electrical activity of the brain varies in frequency and voltage depending on the person's age, state of mental alertness, and health of the underlying brain. However, this activity is sufficiently strong as to be easily measurable in nearly all individuals.

For the purposes of detecting a stroke, embodiments of the present invention place electrodes in strategic locations on the scalp corresponding to the distributions of the arteries supplying the brain, as well as in “eloquent”, important regions of the brain that can cause debilitation if effected by a stroke. This helps to improve stroke reporting over these key areas.

As shown in the side view of FIG. 2A, electrodes are arranged in a roughly straight line position in one embodiment, allowing them to be used with a headband device or other type of protective covering headpiece, as described subsequently. In one embodiment, the electrode arrangement is as follows:
(i) an anterior (front) electrode is situated a few centimeters above the eye, to provide evidence of obstruction of blood flow to the Anterior Cerebral Artery;
(ii) a second electrode is positioned over “Broca's Area” to detect obstruction of blood flow to the expressive language center;
(iii) a third electrode is placed over the Central Gyrus, to detect obstruction of blood flow to the motor and sensory cortexes;
(iv) a fourth electrode is positioned over “Wernicke's Area” to detect obstruction of blood flow to the receptive language center in the superior temporal lobe;
(v) a final, posterior (back) electrode is seated over the occipital pole to detect obstruction of blood flow to the visual cortex.

Significantly, each of the positions given in (i) through (v) above are “vascular points” or more generally “vascular distributions” or “vascular regions”, as the phrase would be understood by one skilled in the art of EEG use and interpretation. While not necessarily limited to signal sensing precisely at vascular distributions, the apparatus and methods of the present invention take advantage of the significant diagnostic information relevant to a stroke condition that is available at these sites. Moreover, the electrodes are paired in the embodiments shown, enabling facile comparison of signals related to vascular behavior; however, alternate embodiments may not require strict left-right pairing of electrodes, but may even use only a single electrode. In addition to the electrode arrangements described, some type of signal ground electrode or attachment (not shown) to the patient may be needed.

It is instructive to note that the placement arrangement shown in the examples of FIGS. 2A-2D differs from the conventional placement used for EEG measurement. As has been emphasized, the conventional EEG pattern is optimized for examining the spread of epileptic electrical activity across the surface of the brain, rather than to observe activity changes that occur in the event of an artery blockage. A typical seizure arrangement montage used with an EEG calls for 21 surface electrodes, covering broad areas of the surface of the brain. Proper placement of each electrode adds to time and complexity for setting up the device, along with increasing the likelihood of electrode or lead failure and likelihood that an individual lead will fall off the skin.

In contrast to the conventional EEG arrangement, embodiments of the present invention place only a few electrodes on relevant vascular points and/or on more “eloquent” regions which, if damaged, would leave the patient with a notable deficit, such as those vascular distributions listed in (i)-(v) above. Thus, embodiments of the present invention focus on obtaining measurements that are more relevant to detecting stroke onset than are the measurements conventionally used with EEG equipment. In addition to having the potential to be more effective for detecting a stroke as it is occurring, embodiments of the present invention require significantly fewer electrodes and leads than does the typical EEG lead configuration used for seizure detection and sleep studies. This reduction in number of leads helps to make it more feasible to design a portable or wearable stroke monitor. Using a more limited number of electrodes, for example, ten electrodes as shown in FIGS. 2A and 2B or four as shown in FIGS. 2C and 2D, specifically placed where they would be most sensitive for picking up a loss of blood flow through one of the major cerebral arteries, enables the device to accurately detect large artery strokes while maintaining an ease of use that allow portable stroke monitoring and detection system 10 to be a wearable patient device.

It should be noted that the electrode configuration listed in (i)-(v) above and generally shown in FIGS. 2A and 2B is one of a number of electrode arrangements that can be used for symmetric pattern sensing, as is described in more detail subsequently. The alternate embodiment of FIGS. 2C and 2D shows an arrangement with only two pairs of electrodes, strategically placed: 53l, 53r and 50l, 50r. In other embodiments using symmetry, as few as one pair of electrodes is used. Other embodiments may use more than five pairs of electrodes, as shown in the example of FIGS. 2A and 2B; however, there are practical limits to what needs to be measured for stroke monitoring as well as to the amount of scalp space available and level of patient discomfort.

### Features to Support Portability

### Headpiece

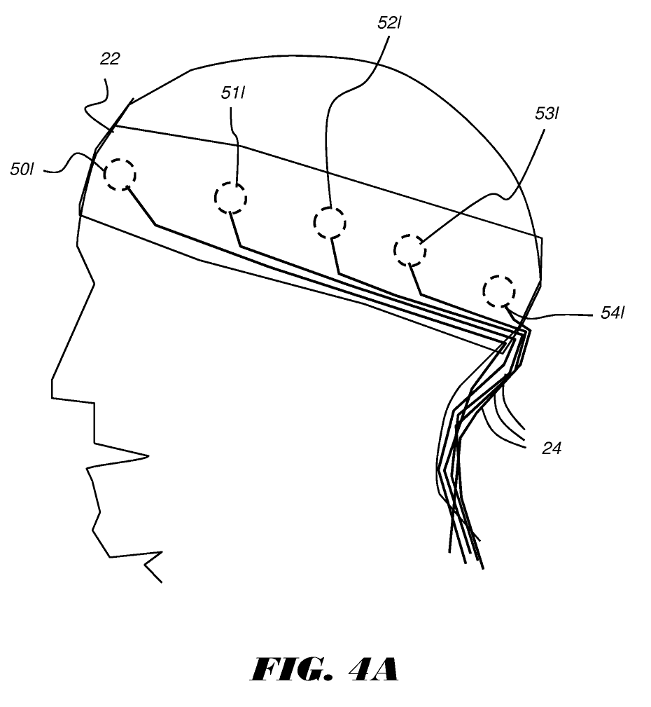

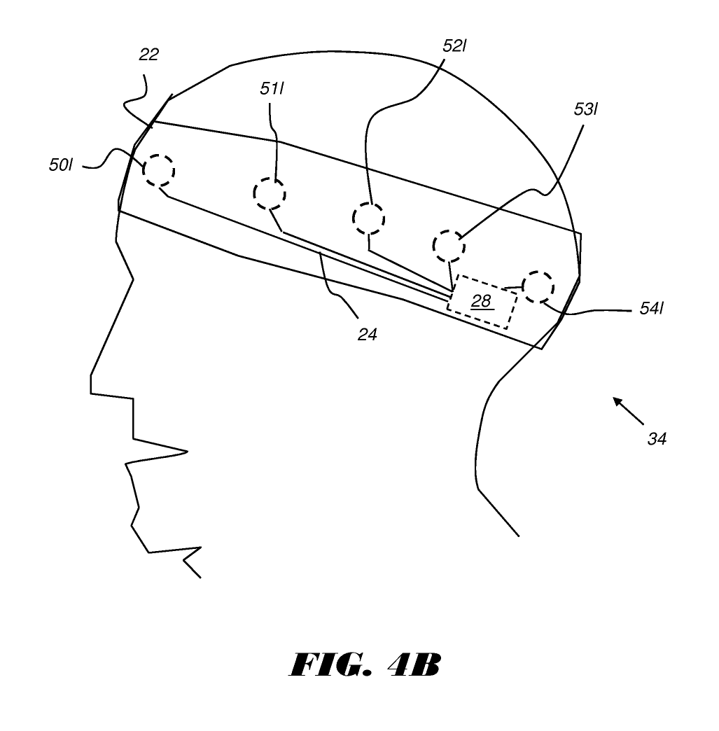

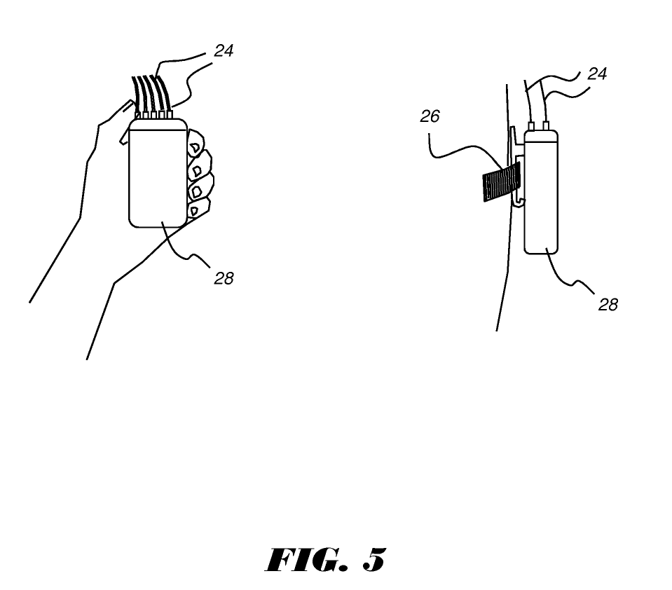

Referring to FIGS. 4A, 4B, and 5, different components of the apparatus that help to provide portable monitoring are shown. An optional headpiece 22 helps to more securely house and protect the electrodes once they are positioned. In the embodiment shown in FIGS. 4A and 4B, headpiece 22 is a headband, similar to that often used for jogging, for example. Using headpiece 22 in this configuration, the electrodes are coupled to the headband in position after electrode placement along the scalp. Electrodes 50l-54r can be adjusted as needed for a particular patient, but have an initial positioning provided for by headpiece 22.

In embodiments of the present invention, electrode 50l-54r placement and attachment along the scalp is first performed by a trained technician or other practitioner, including application of conductive gel and other steps familiar to those skilled in EEG use. Once electrodes are in position, headpiece 22 is fitted over them to conceal and protect electrodes and their attached leads, helping to prevent their inadvertent dislocation during patient movement.

Advantageously, headpiece 22 provides a single unit that can be worn by the patient, as opposed to managing the tangle and confusion of multiple leads 24. The headband or other headpiece 22 can be easily removed and may alternately be used to house the electrodes and leads as a unit and returned to the doctor or monitoring agent. It can be appreciated that headpiece 22 can have any of a number of configurations suited to the needs of particular patients and may be equipped with various padding elements and attachment mechanisms including familiar hook-and-loop fasteners, for example. Headpiece 22 may be given a distinctive coloration for quick identification of a patient who is being monitored or may be designed to be relatively inconspicuous. In alternate embodiments, headpiece 22 is designed as a wig, cap, or other type of device worn on the head of the patient during the monitoring period.

### Monitoring Control

The functions of monitoring apparatus 20 can be executed in a number of ways. As was shown in FIG. 1, an embodiment of portable monitoring and detection system 10 includes a portable monitoring control unit 28 that can be carried by the patient during monitoring or placed near the patient, such as by bedside attachment. FIG. 5 shows a portable monitoring control unit 28 in one embodiment. Monitoring control unit 28 provides a connection for signal communication with each of the paired electrodes that are used for the patient. In the wired version shown in FIG. 5, monitoring control unit 28 can optionally be attached to patient apparel, such as using a strap 26, such as an elastic strap or using belt-and-loop fasteners, a halter strap, belt clip, or other coupling method. Compact packaging allows monitoring control unit 28 to be the size of a hand-held device, as shown in FIG. 5. Further miniaturization allows monitoring control circuit 28 to be a built-in component of headpiece 22 itself, as shown in FIG. 4B, so that leads 24 are not exposed and likely to be inadvertently damaged or loosened.

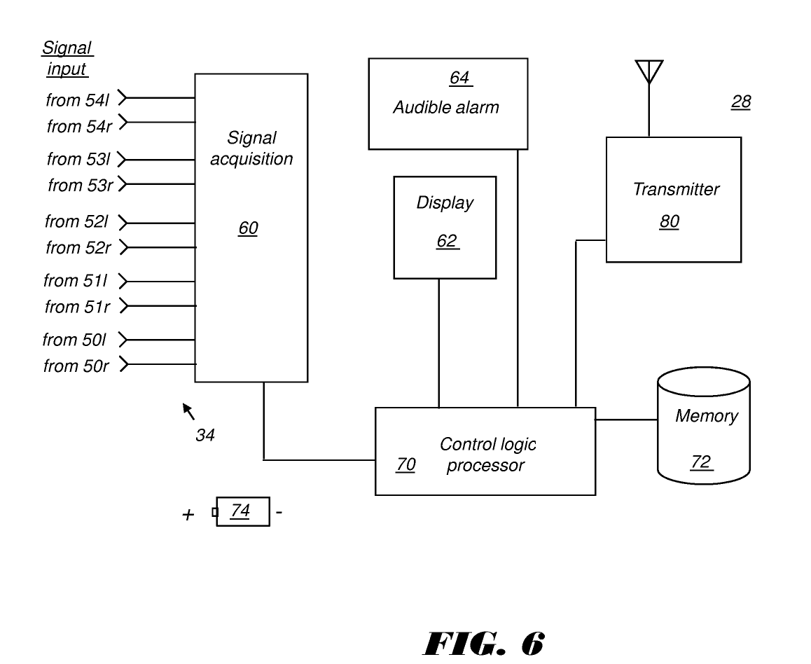

Monitoring control unit 28 can be configured in any of a number of ways, and with varying amounts of on-board logic depending on the overall design of monitoring apparatus 20 of monitoring and detection system 10. The schematic block diagram of FIG. 6 shows internal components of monitoring control unit 28 according to one embodiment. As part of brain wave signal acquisition apparatus 34, a signal acquisition circuit 60 is energizable to obtain the signal from each electrode, using either wired or wireless connection. The electrode signal can be discretely sampled at a high rate or may be continuously sensed as an analog signal.

As is shown in the block diagram of FIG. 6, a control logic processor 70 executes the programmed logic instructions that control and implement signal acquisition, analysis, recording, and reporting functions. In the context of the present disclosure, the term “control logic processor” refers to any of a number of types of dedicated or general purpose logic processors that execute a sequence of stored instructions, including, but not limited to, a computer or host workstation including a networked computer, a microprocessor, a programmable logic array, or other type of logic execution component. The control logic function of monitoring apparatus 20 can be fully contained within an on-board control logic processor 70, but may also advantageously be distributed among two or more processors or executed by or shared with host processor 30 (FIG. 1), for example. An electronic memory 72 supports control logic processor 70 activity, such as by storing programmed instructions. Memory 72 also stores date and time information for stroke detection and may be located on monitoring control unit 28 itself or on a host processor that is in signal communication with monitoring control unit 28, including a remotely networked computer, for example. Memory 72 also provides logic work area, and may also be used for recording information obtained by monitoring control unit 28. This may include information that is stored periodically during the monitoring period, where this information has value for other analysis, for example. This may also include information that is ancillary to stroke detection itself, but may be useful for improving diagnosis, for example.

It should be noted that the term “memory”, equivalent to “computer-accessible memory” or “electronic memory” in the context of the present disclosure, can refer to any type of temporary or more enduring data storage workspace used for storing and operating upon acquired data as well as computed results and accessible to a control logic processor such as a dedicated processor or microprocessor or a computer system, for example. The memory could be non-volatile, using, for example, a long-term storage medium such as magnetic or optical storage. Alternately, the memory could be of a more volatile nature, using an electronic circuit, such as random-access memory (RAM) that is used as a temporary buffer or workspace by a microprocessor or other control logic processor device. Display or print data, for example, is typically stored in a temporary storage buffer that is directly associated with a display or output device and is periodically accessed and refreshed as needed in order to provide displayed or printed data, and may be retained in print or display buffer memory only long enough for providing displayed or printed output, then erased. This temporary storage buffer is also considered to be an electronic memory, as the term is used in the present disclosure. Memory is also used as the data workspace for executing and storing intermediate and final results of calculations and other processing. Computer-accessible memory can be volatile, non-volatile, or a hybrid combination of volatile and non-volatile types.

Referring again to FIG. 6, an optional display 62 can be one or more indicators, such as one or more Light Emitting Diodes (LEDs) that blink or energize in some coordinated pattern to indicate proper ongoing operation or to signal an alarm condition. Display 62 can also be a small panel display, such as a liquid crystal device (LCD) or an Organic LED (OLED) device that is energized to provide text or graphical information relative to patient condition. An optional alarm 64 is provided to emit an audible alarm sound under appropriate conditions. A transmitter 80 provides an output signal to host processor 30 or other remotely located logic device to indicate patient status, warn of a detected stroke, or obtain updated information, for example. A battery 74 provides power for monitoring control unit 28 operation.

In an alternate embodiment, monitoring control unit 28, worn by, carried by, or otherwise disposed near the patient, is configured primarily for brain wave signal acquisition and transmission, without display or alarm functions, and having on-board control logic only sufficient to perform those basic signal acquisition functions. In this alternate embodiment, more complex logic for signal assessment and comparison, warning signal generation, alarm and reporting, recording, and other functions executes remotely from monitoring control unit 28, either through signal communication with a computer or other logic processor located at the same site or through signal communication with a networked computer or other host processor(s) 30 (FIG. 1).

It can be further appreciated that there are a number of ways to implement the signal acquisition, monitoring, recording, and reporting functions of monitoring control unit 28, within the scope of the present invention. For example, logic functions can be shared between control logic processor 70 within control unit 28 and one or more host processors 30 that are in signal communication over a wireless network. Thus, for example, it may not be advantageous for monitoring control unit 28 to perform on-board diagnostic assessment, but rather to provide information to remote host processor 30 on a periodic basis or under certain conditions, such as where signal variation exceeds a predetermined threshold, for example, or a warning condition of some type is detected. Consistent with one embodiment of the present invention, monitoring control unit 28 runs in a monitoring mode until it detects an anomaly such as signal readings that are inconsistent with typical patient brain wave signal patterns or left-right electrode signal imbalance that may indicate a problem. Then, monitoring control unit 28 switches to a reporting mode, in which it acts primarily as a transmitter, directing further signal readings to a remote computer or host processor. This arrangement can be useful, for example, for patients who may exhibit brain wave signal patterns that do not fit conventional or normative models. This can also allow monitoring control unit 28 to be more compact and simpler in design than the more full-fledged unit described with reference to FIG. 6.

Transmitter functions can utilize a cellular phone, wireless networking device, or other wireless transmission device or multi-function device that is separate from monitoring control unit 28. In one embodiment, the device utilizes GPS or other technology for detecting a patient's location to aid a monitoring agent in rapidly locating a patient. This response may include dispatching an ambulance or other emergency responder to the location of the patient.

Referring again to the embodiment of FIG. 6, on-board memory 72 is used to maintain an electronic record of the event, with a time-stamp record, for example. The full record of the event can be subsequently downloaded from monitoring control unit 28.

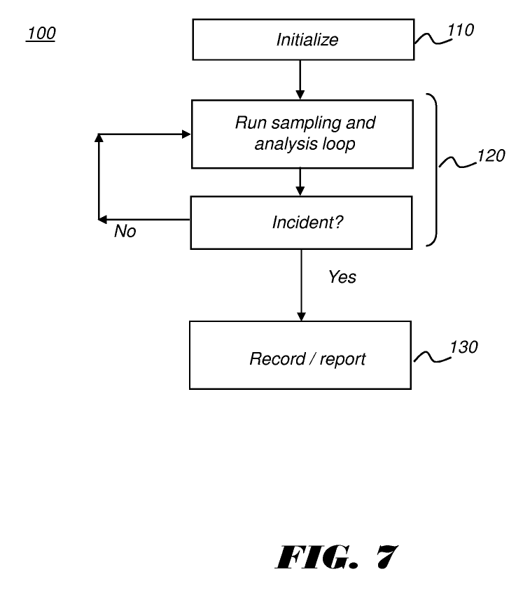

The logic flow diagram of FIG. 7 lists a monitoring sequence 100 that uses monitoring control unit 28 or its equivalent. In an initialization step 110, electrodes are positioned and the monitoring sequence is initialized, such as by calibrating the device to the characteristic brain wave signal behavior of the individual patient, for example. This can require some time period, such as a few minutes or more, for monitoring and “learning” characteristic patient brain wave signal patterns for each placed electrode. This step helps to adapt or condition device response to a particular patient and to reduce the number of false alarms. This conditioning of sensed signal data can be of particular value for patients who already exhibit some left-right signal discrepancies, such as due to a previous stroke, for example.

Continuing with the sequence of FIG. 7, a sampling and analysis loop 120 then obtains the signal from each of the one or more electrodes and tests for overall signal symmetry or for brain wave signal patterns that are not characteristic for the particular patient. As has been noted earlier, sampling and analysis steps can be executed on monitoring control unit 28 or, more generally, at two or more locations in monitoring and detection system 10. Thus, for example, monitoring control unit 28 worn by the patient may be responsible for signal acquisition and transmission, performing the functions of brain wave signal acquisition apparatus 34. Subsequent signal pattern analysis, the function of monitoring apparatus 20, is then performed by host processor 30 (FIG. 1) or other computer system. In sampling and analysis loop 120, a first brain wave signal pattern from the patient is compared against a second brain wave signal pattern from the patient. Where symmetry is used, both first and second brain wave signal patterns are acquired simultaneously. Where deviation from the patient's characteristic brain wave signal patterns is used, the earlier or first acquired brain wave signal serves as the baseline signal, the later or second brain wave signal is compared against this baseline signal to determine whether or not any detected deviation is within normal bounds. If an incident is detected, a reporting step 130 executes, in which date and time information are saved by storage in an electronic memory as a time-stamp record along with any other helpful data and the stroke incident is reported by generating and using a warning signal, such as by emitting an audible sound or by transmitting appropriate data or an alert message to a physician or care facility or to a response unit or other monitoring agent. Consistent with one embodiment of the present invention, reporting is done locally as well as remotely, with the reporting signal utilized so that the patient or local care-giver is alerted at the same time that a remote medical facility or response unit is notified of the stroke event.

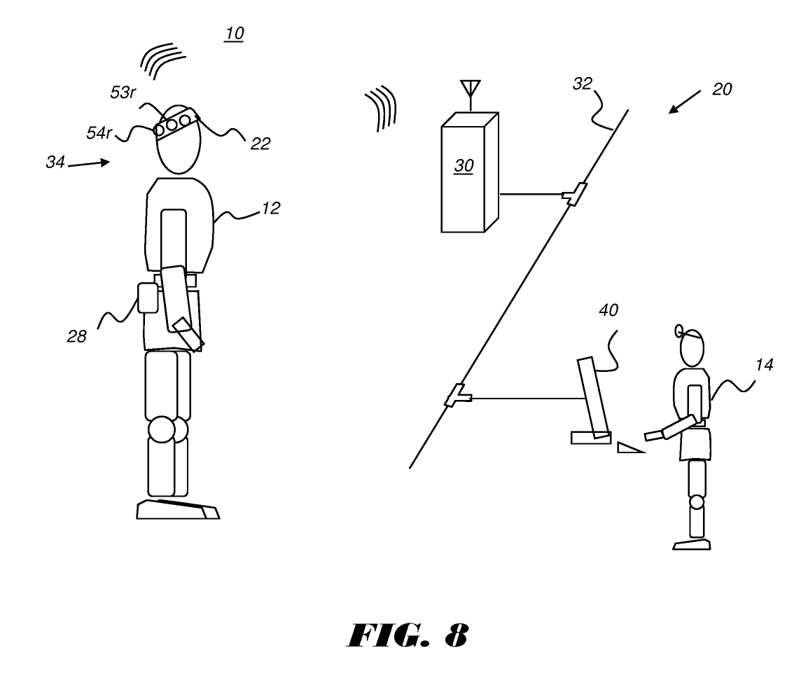

It can be appreciated that there are a number of alternate embodiments of portable monitoring and detection system 10 that can be envisioned. Referring to FIG. 8, for example, there is shown a fully wireless system in which electrodes (53r and 54r shown) communicate wirelessly to transfer signal data either to monitoring control unit 28 or directly to remote host processor 30. In an alternate embodiment, as has been described, monitoring control unit 28 does not analyze signals itself, but, carrying out the functions of brain wave signal acquisition apparatus 34, provides data on a frequent basis to remote host processor 30 that is equipped to do the needed signal analysis and to report and record the stroke incident. The lead placement, recording mechanism, type of electronic monitoring device, method of communications used, and type of alerting system may vary according to the desired implementation of monitoring and detection system 10.

In one embodiment of the present invention, such as for in-hospital monitoring for one or more patients, monitoring control unit 28 is in signal communication, such as in wireless signal communication, with a device recording the patient's brain waves, such as a fixed-based monitoring machine. The fixed based monitoring machine would be equipped with the detection algorithm software. If an event is detected, such a device would generate a signal to alert nearby medical staff and, alternately, the patient, such as by actuating a high volume audible alarm or by triggering a hospital's standard alerting system. This monitoring machine could also keep an electrical and paper record of the event. This model embodiment would be useful, for example, where a patient is monitored at a hospital's Stroke Unit for forty-eight hours or other suitable time period following a TIA event. To use the stroke monitor for hospital use, the electrode wires from the headband unit are connected to monitoring control unit 28 as a bedside monitor, as opposed to connection to a device worn by the patient. Should the bedside monitor detect a notable change in the symmetry of electrode signals, or a significant change from a baseline pattern for the patient, the device provides a warning, such as a loud, audible alarm to alert the patient care team. A “stroke code” is called, and thrombolytic can be administered within minutes. This example embodiment would be somewhat similar to a bedside cardiac arrhythmia monitor found in many hospital intensive care units.

In an alternate embodiment, monitoring control unit 28 as a portable recording device sends its recorded data locally via wireless transmission to a fixed based monitoring machine. This can be used at a nursing station in stroke unit of a hospital. The fixed based monitoring machine is equipped with the detection algorithm software, and an event would trigger a signal to the hospital's alerting system. This alert could include a high volume audible alarm to notify nearby medical staff. This monitoring machine could also keep an electronic memory and paper record of the event. This example would be somewhat similar to a telemetry cardiac monitor found in many hospital's “telemetry units”.

In yet another alternate embodiment, monitoring control unit 28 as a portable recording device transmits data wirelessly to a centralized recording station, equipped with fixed base monitoring processors that execute the detection algorithm software, or monitored by personnel who are trained in the reading of the incoming data. The centralized recording station maintains electrical and hard-copy records of any events. This implementation is particularly suited for patients who may have been released from a hospital.

Advantageously, monitoring control unit 28 has the ability to alert medical personnel quickly. This alert would allow appropriate measures such as automatically “calling 911” or calling a “rapid response stroke code” to be taken, and allows the patient to be treated quickly after onset of a stroke. In certain cases, the invention has the potential to create an alert even before there are visible manifestations of typical stroke onset symptoms. Such a warning system would allow medical personnel to have two critical elements that are of value to producing positive medical outcomes in ischemic stroke patients: the precise time of onset for the stroke, and immediate notification that a stroke has occurred. Documented time of onset and earlier warning could substantially improve a stroke victim's chance of receiving appropriate medical care that substantially reduces the impact of the stroke and, in some cases, reverses it entirely.

### Use Example

Embodiments of the present invention would benefit an eligible patient in immediate ways. Patient X is a 75 year old man with a history of high cholesterol and high blood pressure. On Sunday night, he had a spell, witnessed by his wife, of 15 minutes of inability to formulate words and use his right arm. These symptoms resolved on his way to the hospital. At the hospital, his doctors decide to admit him for a 24 hour observation stay. He does fine clinically, although a 50% tightening of his left carotid artery is discovered.

Since he is at high risk of stroke in the next few weeks, his doctors choose to observe him using monitoring and detection system 10 of the present invention, rather than taking a chance that his stroke is not witnessed. A technician places some adhesive, conducting gel on ten strategic spots on his scalp, corresponding to vascular areas near the main outflow of the large cerebral arteries and particularly “eloquent” regions of the brain, meaning regions which, if damaged, would leave the patient with a notable deficit. Electrodes are then applied to the scalp over the gel. These are clipped into position on his head by the use of a simple headband. Each electrode conducts the small changes in electrical polarity at the scalp, via wire, to a small monitoring apparatus within the headband or worn by the patient. The wires run through a small sleeve in the headband, to the back of the patient's neck, where they are hidden from sight under his shirt until they reach the monitoring device. The patient is discharged from the hospital with instructions to wear the device for one week and the caregiver is given instructions for operating it.

At 1:00 am Tuesday morning a blood clot travels from the patient's left internal carotid artery to his left middle cerebral artery. Due to the lack of blood flow, the brain in that region has a substantial slow down and decrement in the amount of electrical discharges that it creates. Monitoring control unit 28 quickly detects that his left brain cortex has shown a marked difference from the corresponding signals on the right side. Once detected, monitoring control unit 28 sends a signal to an audible alert mechanism on the device, as well as a wireless, “pager quality” signal to a detection center. His wife is awakened by the audible alert and the phone ringing from the call coming from the detection center. The patient's wife answers and confirms that the patient is difficult to arouse, and that he is not speaking to her. An emergency unit is immediately called and an ambulance dispatched. The patient arrives in the emergency room at 1:25 am where his doctors quickly recognize an acute stroke, confirm with the monitoring center that the time of onset was 1:00 am, and administer a strong thrombolytic medicine that breaks up the clot. His symptoms largely resolve. He is left only with a very subtle difficulty in speech, and 95% function of the right arm and is able to walk without any assistance.

Without the invention, this patient would be discharged to home with instructions to his wife that she should “watch him closely”. When the same stroke occurs 1 AM Tuesday morning, he and his wife are both asleep. He wakes up at 8 AM with an observable neurological deficit and the stroke his identified for the first time since his stroke was entirely painless. His wife calls 911, and he is taken to the hospital where his doctors face limited options. They decide that he can only be given aspirin to help dissolve the clot, since it is unclear when the stroke started and it would be too risky to give him thrombolytic medicines such as TPA. He is left permanently disabled with no immediate course of treatment. Thus, it can be appreciated that the apparatus and methods of the present invention offer the potential to significantly improve stroke monitoring and treatment for at-risk patients.

The invention has been described in detail with particular reference to certain preferred embodiments thereof, but it will be understood that variations and modifications can be effected within the scope of the invention as described above, and as noted in the appended claims, by a person of ordinary skill in the art without departing from the scope of the invention.

### PARTS LIST

10. Monitoring and detection system
12. Patient
14. Practitioner
20. Monitoring apparatus
22. Headpiece
24. Electrode lead
26. Strap
28. Monitoring control unit
30. Host processor
32. Network
34. Brain wave signal acquisition apparatus
36, 37. Brain wave signal pattern
40. Display
50l, 50r, 51l, 51r, 52l, 52r, 53l, 53r, 54l, 54r. Electrode
60. Signal acquisition circuit
62. Display
64. Alarm
70. Control logic processor
72. Memory
74. Battery
80. Transmitter
100. Monitoring sequence
110. Initialization step
120. Sampling and analysis loop
130. Reporting step

1. A method for monitoring a patient to detect the onset of a stroke,
the method executed at least in part by a control logic processor and comprising: obtaining, from one or more electrodes on the patient's scalp, at least a first brain wave signal pattern and a second brain wave signal pattern from the patient; comparing at least the first brain wave signal pattern to the second brain wave signal pattern;
and
reporting a stroke and storing, in an electronic memory, a record indicating the time of the stroke according to the comparison.

2. The method of claim 1 wherein the first brain wave signal pattern is obtained from the patient during a first time interval and is stored in the electronic memory as a characteristic brain wave signal pattern for the patient and wherein the second brain wave signal pattern is obtained during a second time interval, later than the first time interval.

3. The method of claim 1 wherein obtaining the first and second brain wave signal patterns comprises obtaining brain wave signals from each of one or more electrode pairs, wherein, each electrode pair obtains a left-side electrode signal from a first electrode on the left side of a patient's scalp and a right-side electrode signal from a second electrode on the right side of the patient's scalp, and wherein the first and second electrodes are symmetrically positioned with respect to each other.

4. The method of claim 1 wherein obtaining the at least first and second brain wave signal patterns further comprises acquiring signals from a control unit worn by the patient.

5. The method of claim 1 wherein the electronic memory is on a device that is worn or carried by the patient.

6. The method of claim 1 wherein reporting the stroke comprises wirelessly transmitting a message.

7. The method of claim 1 wherein reporting the stroke comprises energizing a display.

8. The method of claim 1 wherein reporting the stroke comprises emitting an audible tone.

9. An apparatus for detecting the onset of an ischemic stroke in a patient, comprising:
a) a brain wave signal acquisition apparatus comprising a signal acquisition circuit that is energizable to provide electrode signals acquired from each of one or more electrode pairs that are attached against the patient's scalp, wherein each electrode pair comprises a left electrode disposed at a first position to sense a left-side signal along the left side of the scalp and a corresponding right electrode symmetrically disposed at a second position to sense a corresponding right-side signal on the right side of the scalp;
and
b) a monitoring apparatus that is in signal communication with the brain wave signal acquisition apparatus and is energizable to compare the provided left-side and right-side electrode signals from each electrode pair and to generate a warning signal according to the comparison.

10. The apparatus of claim 9 wherein the brain wave signal acquisition apparatus further comprises a grounding electrode.

11. The apparatus of claim 9 further comprising a headpiece that covers the one or more electrode pairs.

12. The apparatus of claim 9 wherein the signal communication between the brain wave signal acquisition apparatus and left and right electrodes is wireless.

13. The apparatus of claim 9 wherein communication between the brain wave signal acquisition apparatus and monitoring apparatus is wireless.

14. The apparatus of claim 9 wherein the brain wave signal acquisition apparatus comprises a device that is configured to be carried by the patient.

15. The apparatus of claim 11 wherein the headpiece further comprises a transmitter.

16. The apparatus of claim 9 wherein at least one of the one or more electrode pairs is positioned adjacent to a vascular distribution along the scalp.

17. The apparatus of claim 9 wherein the monitoring apparatus comprises a networked logic processing device.

18. A method for monitoring a patient to detect the onset of a stroke, the method executed at least in part by a control logic processor and comprising:
obtaining at least one pair of brain wave signal patterns from each of one or more electrode pairs, wherein, each electrode pair obtains a left-side electrode signal from a first electrode on the left side of a patient's scalp and a right-side electrode signal from a second electrode on the right side of the patient's scalp, and wherein the first and second electrodes are symmetrically positioned with respect to each other;
comparing the left-side and right side electrode signal patterns;
and
reporting a stroke and storing, in an electronic memory, a record indicating the time of the stroke according to the comparison.

19. The method of claim 18 wherein the step of comparing the left and right side electrode signal patterns further comprises comparing the electrode signal patterns according to characteristic brain wave patterns measured previously from the patient.

20. The method of claim 18 wherein obtaining the at least one pair of brain wave signals further comprises wirelessly transmitting the right and left side electrode signals.
